(19) United States
(12) Patent Application Publication
(10) Pub. No.: US 2017/0134817 A1
(43) Pub. Date: May 11, 2017

(54) VIDEO RECOMMENDATION DEVICE AND SYSTEM, AND METHOD THEREOF

(71) Applicant: BOE TECHNOLOGY GROUP CO., LTD., Beijing (CN)

(72) Inventor: Jing YU, Beijing (CN)

(21) Appl. No.: 15/207,812

(22) Filed: Jul. 12, 2016

(30) Foreign Application Priority Data
Nov. 5, 2015 (CN) 201510746633.1

(51) Int. Cl. H04N 21/482; A61B 5/021; A61B 5/024; H04N 21/431; H04N 21/258; H04N 21/45; H04N 21/84; A61B 5/00; A61B 5/145

(52) U.S. Cl. CPC H04N 21/4826; A61B 5/681; A61B 5/021; A61B 5/02438; A61B 5/14532; A61B 5/14542; H04N 21/25891; H04N 21/4532; H04N 21/84; H04N 21/4312

(57) ABSTRACT

A video recommendation device, system and method. An exemplary video recommendation device comprises: a data acquisition module, configured to acquire a user's preset physiological data and the type of the physiological data; and a video recommendation module, configured to display the recommendation information of an object video relevant to the type of the preset physiological data according to the preset physiological data. The present disclosure recommends an object video relevant to the type of the present user's physiological data to the user based on the user's physiological data. The problems of the video recommendation in the prior art, that the recommended results are relatively simple, and the flexibility and pertinence are relatively low, are solved. The present disclosure has effects of higher flexibility and stronger pertinence.

### CROSS-REFERENCE TO RELATED APPLICATION

This application claims the benefit and priority of Chinese Patent Application No. 201510746633.1 filed Nov. 5, 2015. The entire disclosure of the above application is incorporated herein by reference.

### FIELD

The present disclosure relates to a video recommendation device, system and method especially a video recommendation device, system and method based on physiological data of users.

### BACKGROUND

This section provides background information related to the present disclosure which is not necessarily prior art.

Videos may comprise live videos and history videos. Nowadays users may watch videos through an intelligent television. With the development of video market, User-selectable videos have reached a quite a huge amount and an urgent problem to solve is how to help users to quickly select the video he/she wants to watch. A way of solving this problem is recommending videos to the users.

A video recommendation method in relative technologies firstly acquires the click-through rate of respective video in a historical time period, then acquires the video information (e.g. video content information or video title) of videos ranked in top n (n is a preset positive integer) for the click-through rate and display (e.g. popup) the information of the top n video. A user may select the video he wants to watch based on the information. However, the method fails to recommend information aiming at a user's own characteristic. Therefore, the method has at least the following deficiencies: the recommended results are relatively simple, and flexibility and pertinence are relatively low.

### SUMMARY

This section provides a general summary of the disclosure, and is not a comprehensive disclosure of its full scope or all of its features.

In order to solve the problems that the recommended results are relatively simple, and flexibility and pertinence are relatively low, a video recommendation method, device and system are provided.

According to the first aspect of the present disclosure, a video recommendation method is provided. The method comprising:

acquiring the preset physiological data of a user and the type of the preset physiological data;

displaying the recommendation information of the object video which is relevant to the type of the preset physiological data based on the preset physiological data.

Alternatively, displaying the recommendation information of the object video which is relevant to the type of the preset physiological data based on the preset physiological data comprising:

detecting whether the value of the preset physiological data is in the range of health values;

when the value of the preset physiological data is not in the range of health values, then displaying the recommendation information of an object video relevant to the type of the preset physiological data.

Alternatively, after detecting whether the value of the preset physiological data is in the range of health values, the method further comprising:

when the value of the preset physiological data is not in the range of health values, detecting whether the value of the preset physiological data is in the range of Sub-health values, and wherein, the range of Sub-health values contains a first Sub-range in which the minimum value is bigger than the maximum value of the range of health values, and a second Sub-range in which the maximum value is smaller than the minimum value of the range of health values;

when the value of the preset physiological data is not in the range of Sub-health values, stopping displaying the video.

Alternatively, displaying the recommendation information of an object video relevant to the type of the preset physiological data comprising:

acquiring an object keyword corresponding to the type of the preset physiological data based on the correspondence between the type of the preset physiological data and the keyword;

acquiring an object video with a title or tag containing the object keyword and the tag is used for indicating the video content;

displaying the recommendation information of the object video.

Alternatively, displaying the recommendation information of the object video comprising:

display the recommendation information of the object video in a preset way and the preset way comprises at least one of split screen mode, list mode, bullet screen mode and popup mode.

Alternatively, after detecting whether the value of the preset physiological data is in the range of health values, the method further comprising:

when the value of the preset physiological data is not in the range of Sub-health values, sending a prompt message to a preset terminal, and the prompt message is used for prompting that the user's value of the preset physiological data is not in the range of Sub-health values.

Alternatively, after stopping displaying the video, the method further comprising:

displaying a rest prompt message or medical prompt message.

Alternatively, the preset physiological data contains preset number of a same type physiological data in a preset time period before the present moment.

Alternatively, the preset physiological data contains at least two types of physiological data and

displaying the recommendation information of the object video which is relevant to the type of the preset physiological data based on the preset physiological data comprising:

attaching a weight to each type of physiological data based on the degree of deviation between the physiological data and the range of health values of each type of physiological data from at least two types of physiological data, and the weight is positive correlation with the degree of deviation; and

displaying the recommendation information of the object video relevant to the n types of the physiological data with maximum weights and n is a preset positive integer.

Alternatively, the first type of physiological data in the at least two types of physiological data contains preset number of first type physiological data in a preset time period before the present moment, and the first type physiological data is any type physiological data in the at least two types of physiological data.

Alternatively, after detecting whether the value of the preset physiological data is in the range of health values, the method further comprising:

detecting whether there is a live video broadcasting with a content relevant to the type of a preset physiological data when the value of the preset physiological data is not in the range of health values;

recording the live video and obtaining the video data of the live video when there is a live video broadcasting relevant to the type of a preset physiological data.

Alternatively, before acquiring the preset physiological data of a user and the type of the preset physiological data, the method further comprising:

receiving a user's selection information;

determining at least one type of physiological data corresponding to the selection information as the preset physiological data according to preset correspondences.

According to the second aspect of the present disclosure, a video recommendation device is provided. The device comprising:

data acquisition module, configured to acquire a user's preset physiological data and the type of the physiological data;

video recommendation module, configured to display the recommendation information of an object video relevant to the type of the preset physiological data according to the preset physiological data.

Alternatively, the video recommendation module comprising:

health detection unit, configured to detect whether the value of the preset physiological data is in the range of health values;

type recommendation unit, configured to display the recommendation information of an object video relevant to the type of the preset physiological data when the value of the preset physiological data is not in the range of health values.

Alternatively, the video recommendation module further comprising:

Sub-health detection unit, configured to detect whether the value of the preset physiological data is in the range of Sub-health values when the value of the preset physiological data is not in the range of health values, and the range of Sub-health values contains a first Sub-range in which the minimum value is bigger than the maximum value of the range of health values, and a second Sub-range in which the maximum value is smaller than the minimum value of the range of health values;

display stop unit, configured to stop displaying the video when the value of the preset physiological data is not in the range of Sub-health values.

Alternatively, the type recommendation unit comprising:

keyword acquisition sub-unit, configured to acquire an object keyword corresponding to the type of the preset physiological data based on the correspondence between the type of the preset physiological data and the keyword;

video acquisition sub-unit, configured to acquire an object video with a title or tag containing the object keyword and the tag is used for indicating the video content;

information displaying sub-unit, configured to display the recommendation information of the object video.

Alternatively, information display sub-unit is configured to:

display the recommendation information of the object video in a preset way and the preset way comprises at least one of split screen mode, list mode, bullet screen mode and popup mode.

Alternatively, the video recommendation module further comprising:

prompt message sending unit, configured to send a prompt message to a preset terminal when the value of physiological data is not in the range of Sub-health values, and the prompt message is used for instructing that the value of user's physiological data is not in the range of Sub-health values.

Alternatively, the video recommendation module further comprising:

user prompt unit, configured to display a rest prompt message or a medical prompt message.

Alternatively, the preset physiological data contains preset number of the same type physiological data in a preset time period before the present moment.

Alternatively, the preset physiological data contains at least two types of physiological data,

the video recommendation module is configured to:

attach a weight to each type of physiological data based on the degree of deviation between the physiological data and the range of health values of each type of physiological data from at least two types of physiological data, and the weight is positive correlation with the degree of deviation; and

display the recommendation information of the object video relevant to the n types of the physiological data with maximum weights and n is a preset positive integer.

Alternatively, the first type of physiological data in the at least two types of physiological data contains preset number of first type physiological data in a preset time period before the present moment, and the first type physiological data is any type physiological data in the at least two types of physiological data.

Alternatively, the video recommendation module further comprising:

live detection unit, configured to detect whether there is a live video broadcasting with a content relevant to the type of a preset physiological data when the value of the preset physiological data is not in the range of health values;

live recording unit, configured to recording the live video and obtaining the video data of the live video when there is a live video broadcasting with a content relevant to the type of a preset physiological data.

Alternatively, the device further comprising:

data selection module, configured to:

receive the user's selection information;

determine at least one type of physiological data corresponding to the selection information as the preset physiological data according to preset correspondences.

According to the third aspect of the present disclosure, a video recommendation system is provided. The system comprising a wearable apparatus and a video display terminal, the wearable apparatus is used to acquire a user's physiological data and provide the physiological data to the video display terminal;

the video display terminal comprises any video recommendation device according to the second aspect.

By recommending an object video relevant to the type of the present physiological data to a user based on the user's physiological data, the problems of the video recommendation method in prior art, that the recommended results are relatively simple, and the flexibility and pertinence are relatively low, are solved. The present disclosure has effects of higher flexibility and stronger pertinence.

It should be understood that the above general description and the following detailed description are merely exemplary and explanatory, but not limiting.

Further aspects and areas of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of implementation environment of various embodiments of the present disclosure;

FIG. 2 is a flow diagram of a video recommendation method according to an exemplary embodiment of the present disclosure;

FIG. 3a is a flow diagram of another video recommendation method according to exemplary embodiment of the present disclosure;

FIG. 3b is a flow diagram of displaying recommendation information according to the exemplary embodiment in FIG. 3a;

FIG. 3c is a flow diagram of another displaying recommendation information according to the exemplary embodiment in FIG. 3a;

FIG. 4a is a schematic diagram of a video recommendation device according to an exemplary embodiment of the present disclosure;

FIG. 4b is a schematic diagram of a video recommendation module according to the exemplary embodiment in FIG. 4a;

FIG. 4c is a schematic diagram of another video recommendation module according to the exemplary embodiment in FIG. 4a;

FIG. 4d is a schematic diagram of a type recommendation unit according to the exemplary embodiment in FIG. 4a;

FIG. 4e is a schematic diagram of another video recommendation module according to the exemplary embodiment in FIG. 4a;

FIG. 4f is a schematic diagram of another video recommendation module according to the exemplary embodiment in FIG. 4a;

FIG. 4g is another schematic diagram of another video recommendation module according to the exemplary embodiment in FIG. 4a;

FIG. 4h is a schematic diagram of another video recommendation device according to the exemplary embodiment of the present disclosure.

It should be understand that the above figures and relative description are not meant to limit the scope of the present disclosure in any way, but to explain the concept of the present disclosure to the skilled in the art by reference to specific embodiments.

Corresponding reference numerals indicate corresponding parts or features throughout the several views of the drawings.

### DETAILED DESCRIPTION

Example embodiments will now be described more fully with reference to the accompanying drawings.

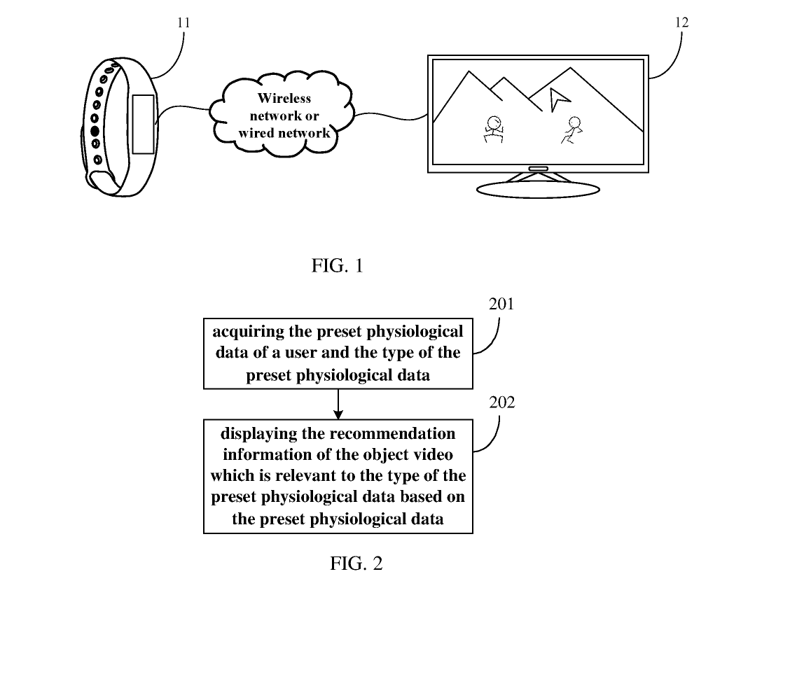

FIG. 1 is a schematic diagram of implementation environment of exemplary embodiments of the present disclosure. The implementation environment may include a wearable apparatus 11 and video display terminal 12.

The wearable apparatus 11 may be a smart bracelet, a smart watch, a pair of smart glasses or a smart necklace, etc. The wearable apparatus 11 may be used to acquire various types of physiological data of users.

The video display terminal 12 may be a television system (including a smart TV or a TV and set-top box), a computer, a cell phone or a tablet PC, etc.

The wearable apparatus 11 and video display terminal 12 may establish a connection be each other via a wireless network or a wired network.

FIG. 2 is a flow diagram of a video recommendation method according to an exemplary embodiment, wherein the video recommendation method performed in the video display terminal 12 in the implementation environment of FIG. 1 is exemplarily showed. The recommendation method may comprise the following steps:

In step 201, acquiring the preset physiological data of a user and the type of the preset physiological data;

In step 202, displaying the recommendation information of the object video which is relevant to the type of the preset physiological data based on the preset physiological data.

As described above, the video recommendation method according to the embodiment of the present disclosure may recommend an object video to a user which is relevant to the type of the physiological data of the present user. The problems of the video recommendation method in prior art, that the recommended results are relatively simple, and the flexibility and pertinence are relatively low, are solved. The present disclosure has effects of higher flexibility and stronger pertinence.

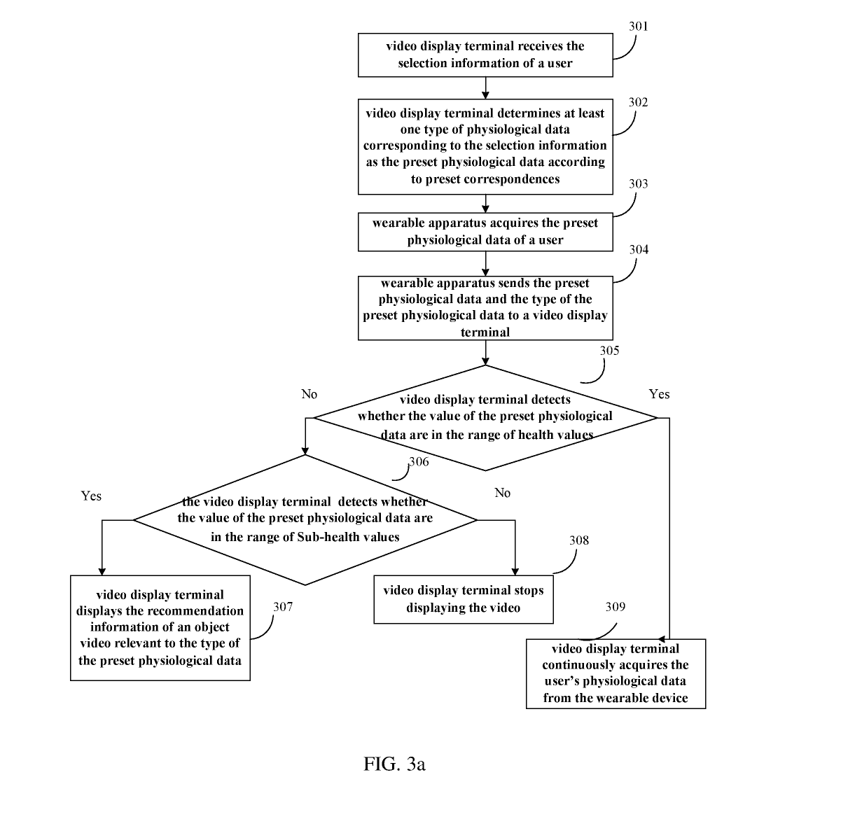

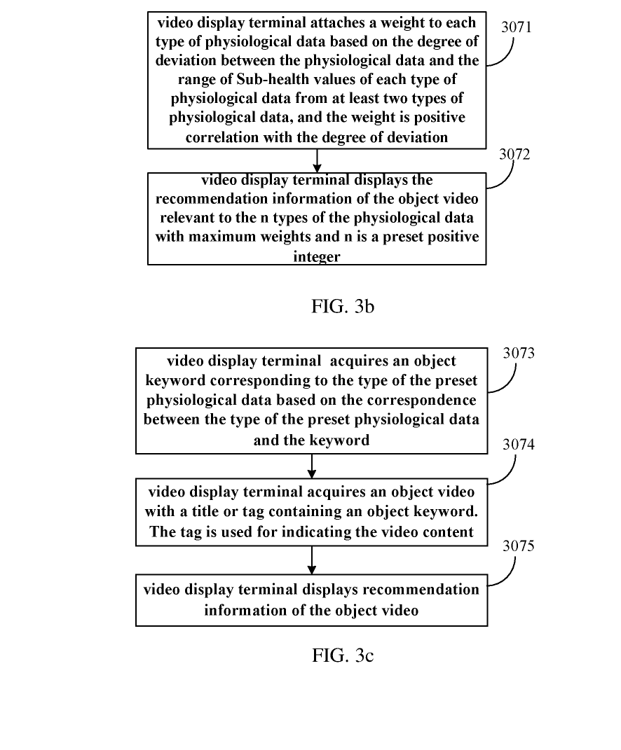

FIG. 3a is a flow diagram of a video recommendation method according to another exemplary embodiment of the present disclosure, wherein the video recommendation method performed in the video display terminal 12 in the implementation environment of FIG. 1 is exemplarily showed. The video recommendation method may comprise the following steps:

In step 301, the video display terminal receives the selection information of a user;

When a user wearing a wearable apparatus watches a video through a video display terminal 12, the selection information of the user is received by the video display terminal 12. The selection information may be used to indicate the video content concerned by the user. For example, if the selection information is some kind of disease selected by the user, this shows the user is more concerned of the videos relevant to this kind of disease (such as the user suffers from this kind of disease).

In step 302, the video display terminal 12 determines at least one type of physiological data corresponding to the selection information as the preset physiological data according to preset correspondences.

When acquiring the selection information, the video display terminal 12 may determine at least one type of physiological data corresponding to the selection information as the preset physiological data according to preset correspondences. And the preset correspondences may record physiological data types according to various selection information. For example, if the selection information is various diseases, the correspondences may be showed as Table 1:

TABLE 1

| Selection Information | Type of Physiological Data |
|---|---|
| hypertension | blood pressure, heart rate |
| diabetes | blood sugar |
| heart disease | blood oxygen, heart rate |

In Table 1, the left column is Selection Information column and the right column is Type of Physiological Data column. The type of physiological data corresponding to the selection information of the user may be acquired. For example, if a user suffers diabetes and concerns video relevant to the disease, he or she may input selection information “diabetes” through a wearable apparatus 11. Then the wearable apparatus 11 acquires that the type of physiological data corresponding to “diabetes” is blood sugar according to Table 1 and then determines “blood sugar” as the preset physiological data. When determining the type of the preset physiological data, the video display terminal 12 sends the determined type information of the preset physiological data to the wearable apparatus 11.

It should be understood that the user's selection information may contains directly the type of the preset physiological data, that is, a user may directly select the type of the preset physiological data.

Moreover, steps 301 and 302 may be performed by a wearable apparatus 11, that is, a user may input selection information through a wearable apparatus 11 and the wearable apparatus 11 determines the preset physiological data.

It should be understood that steps 301 and 302 are steps for determining the type of preset physiological data, while the type of preset physiological data may be set through a wearable apparatus or a video display terminal in the factory without requiring the user's set. That is, steps 301 and 302 are optional steps.

In step 303, a wearable apparatus 11 acquires the preset physiological data of a user.

When determining the preset physiological data, the wearable apparatus 11 may acquire the user's preset physiological data in the current period of time. When the preset physiological data comprises various types of physiological data, the wearable apparatus 11 may acquire the value of various types of physiological data in a period of time. Exemplarily, the preset physiological data comprise heart rate and blood sugar with an acquisition time period of one hour and an acquisition interval of 15 minutes. The preset physiological data acquired by the wearable apparatus 11 may be shown as Table 2:

TABLE 2

| | Time | | | | |
|---|---|---|---|---|---|
| Type | 0 | 15 | 30 | 45 | 60 |
| Blood Rate | 70 | 75 | 85 | 76 | 90 |
| Blood Sugar | 4.1 | 4.5 | 5.6 | 6.1 | 3.9 |

The first line above Table 2 records the times when various types of physiological data are acquired through the wearable apparatus 11. In the situation of Table 2, the wearable apparatus 11 starts from time “0” and acquires physiological data at 15 minutes interval. The unit of heart rate is “Times/minute” and the unit of blood sugar is “M mol/L”.

It should be understood that the wearable apparatus 11 may continuously acquire all the available physiological data, that is, all the available physiological data supported by the hardware of the wearable apparatus 11. Then the wearable apparatus 11 determines one or more types of the physiological data as the preset physiological data according to the type of the preset physiological data selected by the user.

In step 304, the wearable apparatus 11 sends the preset physiological data and the type of the preset physiological data to a video display terminal 12.

When acquires the preset physiological data, the wearable apparatus 11 may send the preset physiological data and the type of the preset physiological data to a video display terminal 12. Exemplarily, the wearable apparatus 11 sends all the data in Table 2 to the video display terminal 12.

It should be understood that the wearable apparatus 11 sends the physiological data to the video display terminal 12 immediately whenever acquires the user's physiological data. For example, in the circumstance of Table 2, the wearable apparatus 11 may acquire the physiological data at 15 minutes interval and immediately send the acquired physiological data to the video display terminal 12.

In step 305, the video display terminal 12 detects whether the value of the preset physiological data is in the range of health values. When the value of the preset physiological data is not in the range of health values, moving to step 306 and when the value of the preset physiological data is in the range of health values, moving to step 309.

When acquires the preset physiological data, the video display terminal 12 may detects whether the value of the preset physiological data is in the range of health values and the range of health values are normal range of user physiological data.

The preset physiological data may comprise various types of physiological data and every type of physiological data in turn, comprises multiple physiological data (as shown in Table 2). For such situation, when detecting preset physiological data, the video display terminal 12 may respectively detect each type of physiological data. It should be understood that the range of health values of each type of physiological data may be the same, or be different.

Exemplarily, taking the data shown in Table 2 as example, the video display terminal 12 may determine respectively whether the two types of physiological data, heart rate and blood sugar, are in the range of health values. When determining whether one type of physiological data is the range of health values, the multiple data of the exact type of physiological data can be combined to reach a determination. For example, when a preset proportion of the physiological data are not in the range of health values, then decide that the type of physiological data are not in the range of health values. Or when preset number of the physiological data deviate from the range of health values, then decide that the type of physiological data are not in the range of health values. The video display terminal 12 may decide whether a type of physiological data are in the range of health values through other way and the present disclosure is not mean to make any restriction. Moreover, determining whether the type of physiological data are in the range of health values through multiple physiological data of the same type in one time period can improve the accuracy of determination.

Moreover, if in the various physiological data, there are one or more types of physiological data outside the range of health values, the video display terminal 12 may take the one or more types of physiological data outside the range of health values as the preset physiological data, and keep performing step 306.

It should be understood that when the value of the preset physiological data is not in the range of health values, the video display terminal 12 may detect whether there is a live video relevant to the type of the preset physiological data to be displayed. And if there is a live video relevant to the type of the preset physiological data to be displayed, acquiring the data stream of the live video and recording the live video, then obtain the video data of the live video. The recorded video data may be recommended to a user when the user uses the video display terminal 12.

Further it should be understood that when the value of the preset physiological data is not in the range of health values, the video display terminal 12 may directly display recommendation information of an object video which is relevant to the type of the preset physiological data. The present embodiments do not make any limitation.

In step 306, the video display terminal 12 detects whether the value of the preset physiological data is in the range of Sub-health values. When the value of the preset physiological data is in the range of Sub-health values, moving to step 307 and when the value of the preset physiological data is not in the range of Sub-health values, moving to step 308.

When detects that the value of the preset physiological data is not in the range of health values, the video display terminal 12 may continue to detect whether the value of the preset physiological data is in the range of Sub-health values. Wherein, the range of Sub-health values contains a first Sub-range in which the minimum value is bigger than the maximum value of the range of health values, and a second Sub-range in which the maximum value is smaller than the minimum value of the range of health values. A user's some types of physiological data are in the range of Sub-health values indicates that the user is in the state of Sub-health. Exemplarily, a range of health values is (50, 60), then the first Sub-range of the range of Sub-health values may be (60, 70) and the second Sub-range may be (40, 50). When there are various types of physiological data in the preset physiological data and in turn, each type of physiological data contains multiple physiological data, the detection procedure may refer to step 305.

It should be understood that steps 306 and 307 may be performed in parallel. In step 305, when it is detected that the value of the preset physiological data is not in the range of health values, step 306 may be performed in parallel with step 307, or step 306 is performed before step 307, or step 307 is performed before step 306. In the situation that steps 306 and 307 may be performed in parallel, step 306 is used for determining whether to stop the display of the video, not for determining whether to display recommendation information, wherein when the value of the preset physiological data is not in the range of Sub-health values, the video display is stopped, while when the value of the preset physiological data is in the range of Sub-health values, the video display is not stopped. The video display terminal 12 may display recommendation information when the video display is stopped.

In step 307, the video display terminal 12 displays the recommendation information of an object video relevant to the type of the preset physiological data.

When determining the preset physiological data is in the range of Sub-health values, the video display terminal 12 may display the recommendation information of an object video relevant to the type of the preset physiological data.

Step 307 comprises the following two situations:

The first situation, the preset physiological data contains various types of physiological data. When displaying the recommendation information of an object video relevant to the type of the preset physiological data, the video display terminal 12 may filter the various types of the physiological data and display the recommendation information of an object video relevant to the filtered type of physiological data. Such that the recommendation effect of the recommendation information is improved.

As shown in FIG. 3b, step 307 may comprise the following two sub-steps:

In sub-step 3071, the video display terminal 12 attaches a weight to each type of physiological data based on the degree of deviation between the physiological data and the range of Sub-health values of each type of physiological data from at least two types of physiological data. The weight is positive correlation with the degree of deviation.

When filtering the various types of physiological data, the video display terminal 12 may firstly attach a weight to each type of physiological data based on the degree of deviation between the physiological data and the range of Sub-health values of each type of physiological data from at least two types of physiological data. The weight is positive correlation with the degree of deviation. Exemplarily, the preset physiological data contains a blood sugar 8 and a heart rate 130, and the range of health values of blood sugar is (3.6, 7) and the range of health values of heart rate is (60, 100). Then the degree of deviation of the blood sugar of the preset physiological data may be (8−7)/7=0.14, and the degree of deviation of the heart rate may be (130−100)/100=0.3, then weights may be attached to each type of the physiological data based on the degree of deviation. When attaching a weight, the product of the degree of deviation and the preset coefficient of the type of the physiological data may be taken as the weight of the type of the physiological data. The preset coefficient of the type of the physiological data may be set with the important degree of the type of the physiological data as a reference. For example, the preset coefficient of heart rate is set higher, as 1, and the preset coefficient of blood sugar is set lower, as 0.7, and then the weight of blood sugar will be 0.7*0.14=0.098 and the weight of heart rate will be 0.3*1=0.3. Moreover, there are other ways for setting a weight and the present disclosure does not make any limitation.

It should be understood that when each type of physiological data contains multiple physiological data of a time period (for example, as shown in Table 2), the average of the multiple physiological data may be taken as the value of the type of physiological data. For example, the average of the blood sugar shown in Table 2 is (4.1+4.5+5.6+6.1+3.9)/6=3.98. There are other ways for determining the value of a certain type of physiological data and the present disclosure does not make any limitation.

In sub-step 3072, the video display terminal 12 displays the recommendation information of the object video relevant to the n types of the physiological data with maximum weights and n is a preset positive integer.

After attaching a weight to each type of physiological data, the video display terminal 12 may display the recommendation information of the object video relevant to the n types of the physiological data with maximum weights, and n is preset positive integer. The displayed recommendation information is relevant to some more severe types of physiological data, and thus enhancing the recommendation efficiency and effect of the recommendation information.

The second situation: the preset physiological data is physiological data of one type.

As shown is FIG. 3c, the step may comprise the following sub-steps:

In sub-step 3073, the video display terminal 12 acquires an object keyword corresponding to the type of the preset physiological data based on the correspondence between the type of the preset physiological data and the keyword.

The video display terminal 12 may acquire the object keyword corresponding to the type of the preset physiological data based on the correspondence between the type of the preset physiological data and the keyword. Each type of the physiological data may be corresponding to multiple keywords, and the video display terminal 12 may acquire at least one keyword corresponding to the type of the preset physiological data. For example, the correspondence between the type of the preset physiological data and the keyword may be as shown in Table 3:

TABLE 3

| Type | Keyword |
|---|---|
| Blood Sugar | Hyperglycemia, diabetes, urine sugar |
| Heart Rate | heart disease, Sinus tachycardia |
| Blood Pressure | Hypertension, cardio-cerebrovascular diseases, |
| | High blood fat, high blood pressure and high blood sugar |

In Table 3, the left column is a type column recoding three types of physiological data. And the right column is a keyword column recording the keywords corresponding to the types of physiological data on the left. The video display terminal 12 may acquire the object keyword corresponding to the type of the preset physiological data based on the table.

In sub-step 3074, the video display terminal 12 acquires an object video with a title or tag containing an object keyword. The tag is used for indicating the video content.

The video display terminal 12 may acquire an object video with a title or tag containing an object keyword from a preset video library after obtaining the keyword corresponding to the type of the preset physiological data. The tag is used for indicating the video content. Normally the title or tag of a video may contain description information of the video content. The video display terminal 12 may take the video in the preset video library with a title or tag containing an object keyword as the object video. Herein the video may be a live video or video stored in a server. The server may provide a preset video library and a programme of videos in the preset video library.

In sub-step 3075, the video display terminal 12 displays recommendation information of the object video.

The video display terminal 12 may display recommendation information of an object video after determining the object video. The recommendation information may be the title, tag or screenshot of the recommendation information.

The above two situations may be combined to be carried out. For example, the recommendation information of the object video relevant to multiple types of physiological data in the first approach is displayed through the recommendation information display method in the second approach.

Further, when displaying recommendation information, the video display terminal 12 may display the recommendation information of an object video in a preset way. The preset way comprises at least one of split screen mode, list mode, bullet screen mode and popup mode.

In step 308, the video display terminal 12 stops displaying the video.

When the value of physiological data is not in the range of Sub-health values, it shows that the user is in poor health condition and does not suitable to continue to watch the video. The video display terminal 12 may stop displaying the video and then present rest prompt message or medical prompt message.

Moreover, when the value of physiological data is not in the range of Sub-health values, the video display terminal 12 may send prompt message to a preset terminal. The prompt message is used for prompting that the user's value of physiological data is not in the range of Sub-health values. The preset terminal may be other user's terminal and the other user may be the family member or doctor of the user of the preset terminal (such that the other user may directly reminder the user of the wearable device).

It should be understood that the when the value of physiological data is not in the range of Sub-health values, the video display terminal 12 may control the wearable device 11 to prompt the user to stop watching the video via vibrating or making a noise.

In step 309, the video display terminal 12 continuously acquires the user's physiological data from the wearable device 11.

When the value of physiological data is in the range of health values, it shows that the user is in good health condition he video display terminal 12 continuously acquires the user's physiological data from the wearable device 11, in order to continuously monitor the user's health condition.

Alternatively, the video recommendation method provided by the present disclosure determines whether the physiological data of a type is normal through multiple physiological data of one type in a time period, so as to achieve the effect of improving the accuracy of determination.

Alternatively, the video recommendation method provided by the present disclosure filters n types of the physiological data according to the degree of deviation between the various types of physiological data and the range of health values, and then displays the recommendation information of an object video relevant to the n types of physiological data, so as to achieve the effects of reducing the amount of the recommendation information and improving the recommendation efficiency of the recommendation information.

Alternatively, the video recommendation method provided by the present disclosure stops displaying a video when the user's physiological data is not in the range of health values and the range of Sub-health values, so as to achieve the effects of ensuring that the user is in good health and avoiding further deterioration of the user's body.

Alternatively, the video recommendation method provided by the present disclosure recommending relevant videos when the user's physiological data is in the range of Sub-health values, so at to achieve the effect of improving the user's body healthy through video recommendation.

As described above, the video recommendation method provided by the present disclosure recommends a objective video relevant to the type of the present user's physiological data according to the user's physiological data, such that the problems in the prior art that the recommended results are relatively simple, and the flexibility and pertinence are relatively low, are solved. The present disclosure has effects of higher flexibility and stronger pertinence.

The following is the video recommendation method provided by the present disclosure, suitable for performing the method embodiments of the present disclosure.

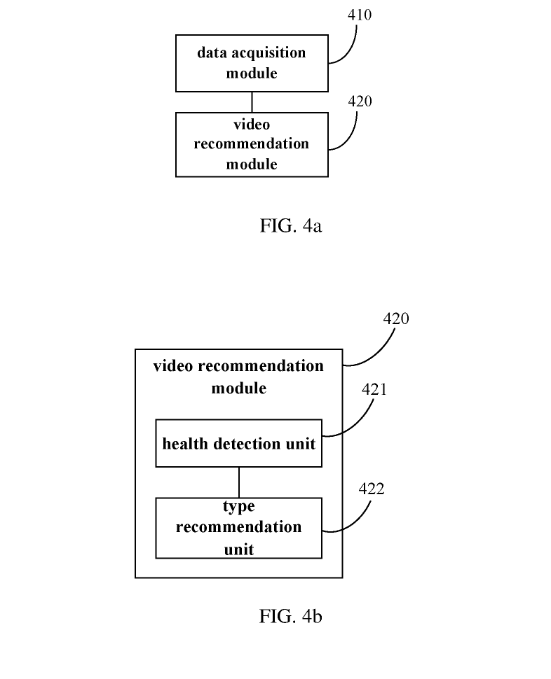

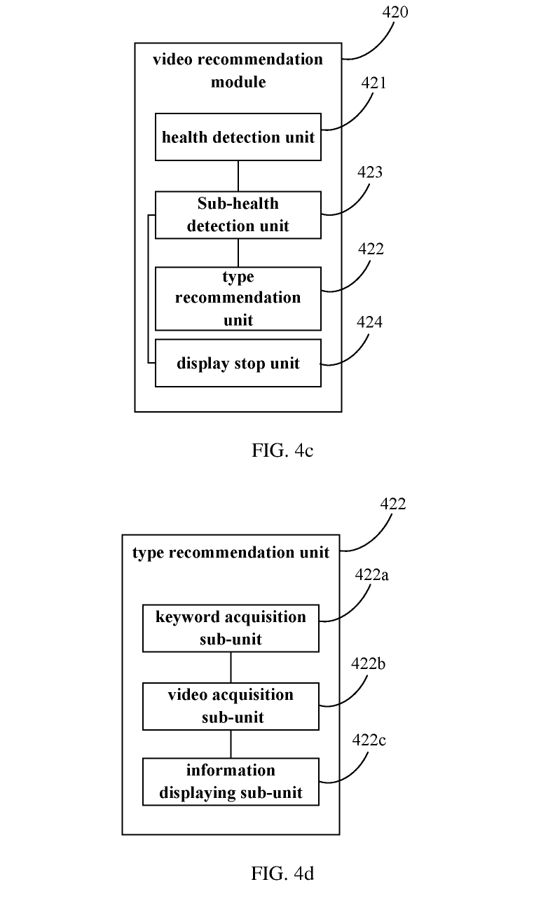

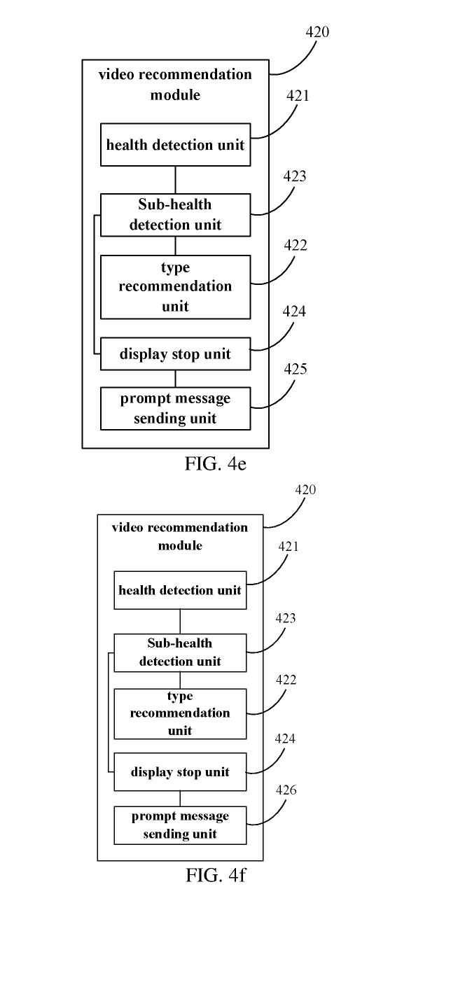

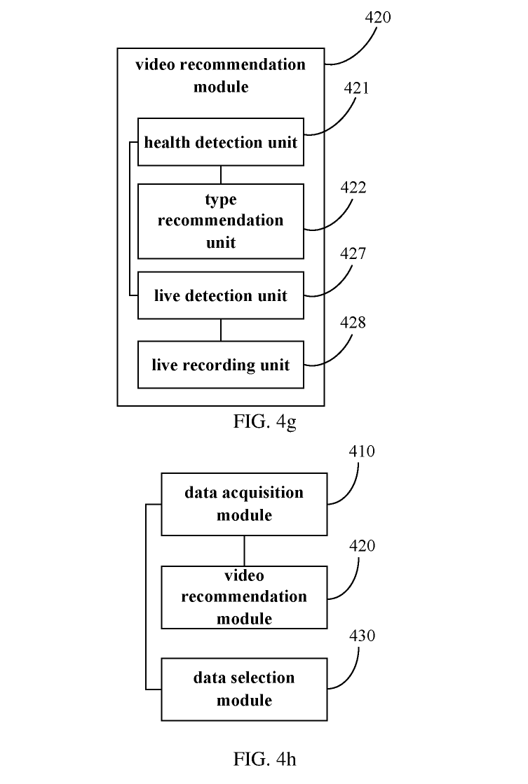

FIG. 4a is a schematic diagram of a video recommendation device according to an exemplary embodiment of the present disclosure. The video recommendation device may comprise:

data acquisition module 410, configured to acquire a user's preset physiological data and the type of the physiological data;

video recommendation module 420, configured to display the recommendation information of an object video relevant to the type of the preset physiological data according to the preset physiological data;

As described above, the video recommendation device provided by the present disclosure recommends a objective video relevant to the type of the present user's physiological data according to the user's physiological data, such that the problems in the prior art that the recommended results are relatively simple, and the flexibility and pertinence are relatively low, are solved. The present disclosure has effects of higher flexibility and stronger pertinence.

Alternatively, as shown in FIG. 4b, the video recommendation module 420 comprises:

health detection unit 421, configured to detect whether the value of the preset physiological data is in the range of health values;

type recommendation unit 422, configured to display the recommendation information of an object video relevant to the type of the preset physiological data when the value of the preset physiological data is not in the range of health values.

Alternatively, as shown in FIG. 4c, the video recommendation module 420 further comprises:

Sub-health detection unit 423, configured to detect whether the value of the preset physiological data is in the range of Sub-health values when the value of the preset physiological data is not in the range of health values. The range of Sub-health values contains a first Sub-range in which the minimum value is bigger than the maximum value of the range of health values, and a second Sub-range in which the maximum value is smaller than the minimum value of the range of health values;

display stop unit 424, configured to stop displaying the video when the value of the preset physiological data is not in the range of Sub-health values.

Alternatively, as shown in FIG. 4d, the type recommendation unit 422 comprises:

keyword acquisition sub-unit 422a, configured to acquire an object keyword corresponding to the type of the preset physiological data based on the correspondence between the type of the preset physiological data and the keyword;

video acquisition sub-unit 422b, configured to acquire an object video with a title or tag containing an object keyword and the tag is used for indicating the video content;

information displaying sub-unit 422c, configured to display the recommendation information of the object video in a preset way and the preset way comprises at least one of split screen mode, list mode, bullet screen mode and popup mode.

Alternatively, as shown in FIG. 4e, video recommendation module 420 further comprises:

prompt message sending unit 425, configured to send a prompt message to a preset terminal when the value of physiological data is not in the range of Sub-health values, and the prompt message is used for instructing that the value of user's physiological data is not in the range of Sub-health values.

Alternatively, as shown in FIG. 4f, video recommendation module 420 further comprises:

user prompt unit 426, configured to display a rest prompt message or a medical prompt message.

Alternatively, the preset physiological data contains preset number of a same type physiological data in a preset time period before the present moment; and

Alternatively, the preset physiological data contains at least two types of physiological data.

Video recommendation module 420 is configured to:

attach a weight to each type of physiological data based on the degree of deviation between the physiological data of each type of physiological data and the range of health values from at least two types of physiological data, and the weight is positive correlation with the degree of deviation;

display the recommendation information of the object video relevant to the n types of the physiological data with maximum weights, and n is a preset positive integer

Alternatively, the first type of physiological data in the at least two types of physiological data contains preset number of first type physiological data in a preset time period before the present moment, and the first type physiological data is any type physiological data in the at least two types of physiological data.

Alternatively, as shown in FIG. 4g, the video recommendation module 420 further comprises:

live detection unit 427, configured to detect whether there is a live video broadcasting with a content relevant to the type of a preset physiological data when the value of the preset physiological data is not in the range of health values;

live recording unit 428, configured to recording the live video and obtaining the video data of the live video when there is a live video broadcasting with a content relevant to the type of a preset physiological data.

Alternatively, as shown in FIG. 4g, the device further comprises data selection module 430 configured to:

receive selection information from a user;

determine at least one type of physiological data corresponding to the selection information as a preset physiological data according to a preset correspondence.

Alternatively, the video recommendation device provided by the embodiments of the present disclosure determines whether the physiological data of a type is normal through multiple physiological data of one type in a time period, so as to achieve the effect of improving the accuracy of determination.

Alternatively, the video recommendation device provided by the embodiments of the present disclosure filters n types of the physiological data according to the degree of deviation between the various types of physiological data and the range of health values, and then displays the recommendation information of an object video relevant to the n types of physiological data, so as to achieve the effects of reducing the amount of the recommendation information and improving the recommendation efficiency of the recommendation information.

Alternatively, the video recommendation device provided by the embodiments of the present disclosure stops displaying a video when the user's physiological data is not in the range of health values and the range of Sub-health values, so as to achieve the effects of ensuring that the user is in good health and avoiding further deterioration of the user's body.

Alternatively, the video recommendation device provided by the embodiments of the present disclosure recommending relevant videos when the user's physiological data is in the range of Sub-health values, so at to achieve the effect of improving the user's body healthy through video recommendation.

As described above, the video recommendation device according to the embodiments of the present disclosure may recommend an object video to a user which is relevant to the type of the physiological data of the present user. The problems of the video recommendation method in prior art, that the recommended results are relatively simple, and the flexibility and pertinence are relatively low, are solved. The present disclosure has effects of higher flexibility and stronger pertinence.

Moreover, the embodiments of the present disclosure also provides a video recommendation system, the system may comprise a wearable apparatus and a video display terminal.

The wearable apparatus 11 is used for acquiring a user's physiological data and providing the physiological data to the video display terminal 12.

The video display terminal 12 comprises the video recommendation device as shown in FIG. 4a or the video recommendation device as shown in FIG. 4h.

As to the devices according to the above embodiments, the specific ways of each module for performing operations have been described in detail in the embodiments concerning the video recommendation method. And thus specific illustration will not be provided herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A video recommendation device, comprising:
data acquisition module, configured to acquire a user's preset physiological data and the type of the physiological data;
video recommendation module, configured to display the recommendation information of an object video relevant to the type of the preset physiological data according to the preset physiological data.

2. The device according to claim 1, wherein the video recommendation module further comprising:
health detection unit, configured to detect whether the value of the preset physiological data is in the range of health values;
type recommendation unit, configured to display the recommendation information of an object video relevant to the type of the preset physiological data when the value of the preset physiological data is not in the range of health values.

3. The device according to claim 2, wherein the video recommendation module further comprising:
Sub-health detection unit, configured to detect whether the value of the preset physiological data is in the range of Sub-health values when the value of the preset physiological data is not in the range of health values, and the range of Sub-health values contains a first Sub-range in which the minimum value is bigger than the maximum value of the range of health values, and a second Sub-range in which the maximum value is smaller than the minimum value of the range of health values;
display stop unit, configured to stop displaying the video when the value of the preset physiological data is not in the range of Sub-health values.

4. The device according to claim 2, wherein the type recommendation unit further comprising:
keyword acquisition sub-unit, configured to acquire an object keyword corresponding to the type of the preset physiological data based on the correspondence between the type of the preset physiological data and the keyword;
video acquisition sub-unit, configured to acquire an object video with a title or tag containing the object keyword and the tag is used for indicating the video content;
information displaying sub-unit, configured to display the recommendation information of the object video.

5. The device according to claim 1, wherein the preset physiological data contains preset number of the same type physiological data in a preset time period before the present moment.

6. The device according to claim 1, wherein the preset physiological data contains at least two types of physiological data and
the video recommendation module is configured to:
attach a weight to each type of physiological data based on the degree of deviation between the physiological data and the range of health values of each type of physiological data from at least two types of physiological data, and the weight is positive correlation with the degree of deviation; and
display the recommendation information of the object video relevant to the n types of the physiological data with maximum weights and n is a preset positive integer.

7. The device according to claim 6, wherein the first type of physiological data in the at least two types of physiological data contains preset number of first type physiological data in a preset time period before the present moment, and the first type physiological data is any type physiological data in the at least two types of physiological data.

8. The device according to claim 1, further comprising:
data selection module, configured to:
receive the user's selection information;
determine at least one type of physiological data corresponding to the selection information as the preset physiological data according to preset correspondences.

9. A video recommendation system, comprising a wearable apparatus and a video display terminal, the wearable apparatus is used to acquire a user's physiological data and provide the physiological data to the video display terminal;
the video display terminal comprises a video recommendation device, wherein the video recommendation device comprises:
data acquisition module, configured to acquire a user's preset physiological data and the type of the physiological data;
video recommendation module, configured to display the recommendation information of an object video relevant to the type of the preset physiological data according to the preset physiological data.

10. The video recommendation system according to claim 9, wherein the video recommendation module further comprising:
health detection unit, configured to detect whether the value of the preset physiological data is in the range of health values;
type recommendation unit, configured to display the recommendation information of an object video relevant to the type of the preset physiological data when the value of the preset physiological data is not in the range of health values.

11. The video recommendation system according to claim 9, wherein the preset physiological data contains at least two types of physiological data,
the video recommendation module is configured to:
attach a weight to each type of physiological data based on the degree of deviation between the physiological data and the range of health values of each type of physiological data from at least two types of physiological data, and the weight is positive correlation with the degree of deviation; and
display the recommendation information of the object video relevant to the n types of the physiological data with maximum weights and n is a preset positive integer.

12. The video recommendation system according to claim 9, wherein the video recommendation device further comprising:
data selection module, configured to:
receive the user's selection information;
determine at least one type of physiological data corresponding to the selection information as the preset physiological data according to preset correspondences.

13. A video recommendation method comprising:
acquiring the preset physiological data of a user and the type of the preset physiological data;
displaying the recommendation information of the object video which is relevant to the type of the preset physiological data based on the preset physiological data.

14. The method according to claim 13, wherein displaying the recommendation information of the object video which is relevant to the type of the preset physiological data based on the preset physiological data comprising:
detecting whether the value of the preset physiological data is in the range of health values;
when the value of the preset physiological data is not in the range of health values, then displaying the recommendation information of an object video relevant to the type of the preset physiological data.

15. The method according to claim 14, after detecting whether the value of the preset physiological data is in the range of health values, the method further comprising:
when the value of the preset physiological data is not in the range of health values, detecting whether the value of the preset physiological data is in the range of Sub-health values, and wherein, the range of Sub-health values contains a first Sub-range in which the minimum value is bigger than the maximum value of the range of health values, and a second Sub-range in which the maximum value is smaller than the minimum value of the range of health values;
when the value of the preset physiological data is not in the range of Sub-health values, stopping displaying the video.

16. The method according to claim 14, wherein displaying the recommendation information of an object video relevant to the type of the preset physiological data comprising:
acquiring an object keyword corresponding to the type of the preset physiological data based on the correspondence between the type of the preset physiological data and the keyword;
acquiring an object video with a title or tag containing the object keyword and the tag is used for indicating the video content;
displaying the recommendation information of the object video.

17. The method according to claim 13, wherein the preset physiological data contains preset number of a same type physiological data in a preset time period before the present moment.

18. The method according to claim 13, wherein the preset physiological data contains at least two types of physiological data and
displaying the recommendation information of the object video which is relevant to the type of the preset physiological data based on the preset physiological data comprising:
attaching a weight to each type of physiological data based on the degree of deviation between the physiological data and the range of health values of each type of physiological data from at least two types of physiological data, and the weight is positive correlation with the degree of deviation; and
displaying the recommendation information of the object video relevant to the n types of the physiological data with maximum weights and n is a preset positive integer.

19. The method according to claim 18, wherein the first type of physiological data in the at least two types of physiological data contains preset number of first type physiological data in a preset time period before the present moment, and the first type physiological data is any type physiological data in the at least two types of physiological data.

20. The method according to claim 13, wherein before acquiring the preset physiological data of a user and the type of the preset physiological data, the method further comprising:
receiving the user's selection information;
determining at least one type of physiological data corresponding to the selection information as the preset physiological data according to preset correspondences.
